(19) United States
(12) Patent Application Publication
(10) Pub. No.: US 2021/0318311 A1
(43) Pub. Date: Oct. 14, 2021

(54) SIMULTANEOUS DETECTION OF HUMORAL AND INFLAMMATORY BIOMARKERS

(71) Applicant: MBio Diagnostics, Inc., Boulder, CO (US)

(72) Inventor: David Okrongly, Boulder, CO (US)

(21) Appl. No.: 17/229,512

(22) Filed: Apr. 13, 2021

Related U.S. Application Data
(60) Provisional application No. 63/009,412, filed on Apr. 13, 2020.
(60) Provisional application No. 63/009,908, filed on Apr. 14, 2020.

(51) Int. Cl. G01N 33/569; G01N 21/64; G01N 33/68

(52) U.S. Cl. CPC G01N 33/56983; G01N 21/6428; G01N 33/6854; G01N 2021/6439; G01N 2333/165; G01N 2800/60; G01N 2333/525; G01N 2333/4737; G01N 2333/585; G01N 2800/56; G01N 2333/5412

(57) ABSTRACT

The present disclosure provides a rapid test for determining state of an infection (e.g., a coronavirus such as Covid-2019) in a subject. The test may include the steps of detecting of an antibody specific to the pathogen in a blood sample from the subject, and detecting and quantitating the level of at least one inflammatory biomarker in the same subject.

### CROSS-REFERENCE TO RELATED APPLICATIONS

This application claims benefit of priority to U.S. Provisional Patent Application No. 63/009,412 filed on Apr. 13, 2020, and to U.S. Provisional Patent Application No. 63/009,908 filed on Apr. 14, 2020, both of which are incorporated herein by reference in their entirety.

### BACKGROUND

In a viral pandemic where community spread is primarily realized through inhalation of airborne aerosols, an effective strategy to decrease community spread is through government enforced quarantines and shelter in place orders, as well as practice of social distancing and use of personal protective equipment (PPE). This has played out in recent times with the COVID-19 global pandemic. This disclosure provides a means to test individuals and determine when these measures can be safely ended for an individual.

The SARS-CoV-2 virus that causes the COVID-19 disease presents with a broad range of symptoms, from patients who experience very minor or no symptoms, to very serious cases that may lead to hospitalization. Chen et al., Lancet, 395:507-13 (2020). Studies on COVID-19 and other influenza-like illnesses (ILI) have demonstrated the important relationship between certain white blood cell and host inflammatory response markers as prognostic indicators of acute respiratory distress syndrome (ARDS) and death. The host inflammatory response markers include pro-inflammatory cytokines and acute-phase proteins, among others. In viral diseases, these host inflammatory response markers rapidly return to pre-infection levels in convalescing patients.

Further consequence of the host inflammatory response is the creation of a protective cellular and humoral memory that can spring into action in the event of any future encounters with a similar pathogen. Humoral response can be readily detected by the presence of antigen-specific immunoglobulins in the blood (also known as antibodies) that can be reactive to both surface and interior viral proteins. Antibodies may target and/or neutralize the virus, and may help recruit other immune cells to fight against the virus. These immunoglobulins are typically the IgG and IgM classes, but may also include IgA, particularly secretory IgA (sIgA) in the respiratory tract and body fluids. Tests that measure for the presence of immunoglobulins against a pathogen are known as serology tests. The presence of these antibodies is indicative of exposure to an infectious agent and may be quantitatively measured to indicate protection against future infection.

In the wake of an epidemic or pandemic, public health officials will usually rely on a time-based approach for determining when a person is safe to return to normal activity and social contact. However, up to four weeks from disease onset, a person who appears to have recovered from a viral infection may still have active viral shedding and may still be infectious to other healthy individuals. Currently, no test is available that will allow people who are low risk for transmitting virus to return to normal life as soon as possible, and may also be informed whether or not they have protective antibodies. This is particularly important for healthcare workers, teachers, first responders and other occupations where frequent close community contact is normal. This disclosure provides a solution to address the above concerns, which is to perform two tests either simultaneously or one test after the other at the same location: the first test is to determine whether a patient has antibodies against the virus and is safe from becoming infected again; the second test is to determine whether the patient has an active infection and could potentially spread the virus to others. If the patient has an active infection or if he/she does not have antibodies against the virus, a clinician may recommend to the patient that he/she should continue to be isolated or quarantined. The second test may be performed by PCR. However, because a PCR test is difficult to perform, it's availability may be restricted to a few high complexity clinical labs, and test results may not be available for several days. By contrast, the instant disclosure provides a new alternative method to achieve the same goal in minutes at even remote locations.

### BRIEF DESCRIPTION OF THE FIGURES

The following figures form part of the present specification and are included to further illustrate aspects of the present invention.

FIG. 1 illustrates an assay for detecting IgG. In one example, SARS-CoV-2 antigen is printed on the Planar waveguide (PWG) surface. Then, the surface is incubated with a patient sample. Then, a wash step may be used to remove excess patient sample. Subsequently, the surface is incubated with a labeling reagent (such as Anti-human IgG-A647 Fluorescent Conjugate). Optionally, another wash step may be performed to remove excess detect antibody. Then, level of IgG against SARS-CoV-2 antigen in the patient sample can be detected by measuring the fluorescence on the surface.

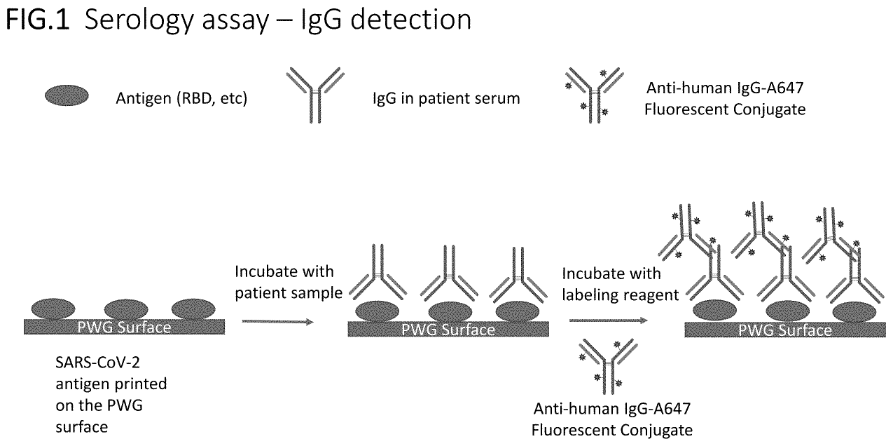

FIG. 2 illustrates an assay for detecting IgM (μ-chain capture). In one example, anti-IgM antibody is printed on the PWG surface. Then, the surface is incubated with a patient sample. Then, a wash step may be used to remove excess patient sample. Subsequently, the surface is incubated with a labeled antigen (such as AF-647 labeled antigen). Another wash step may be used to remove excess labeled antigen. Then, level of IgM in the patient sample can be detected by measuring the fluorescence on the surface.

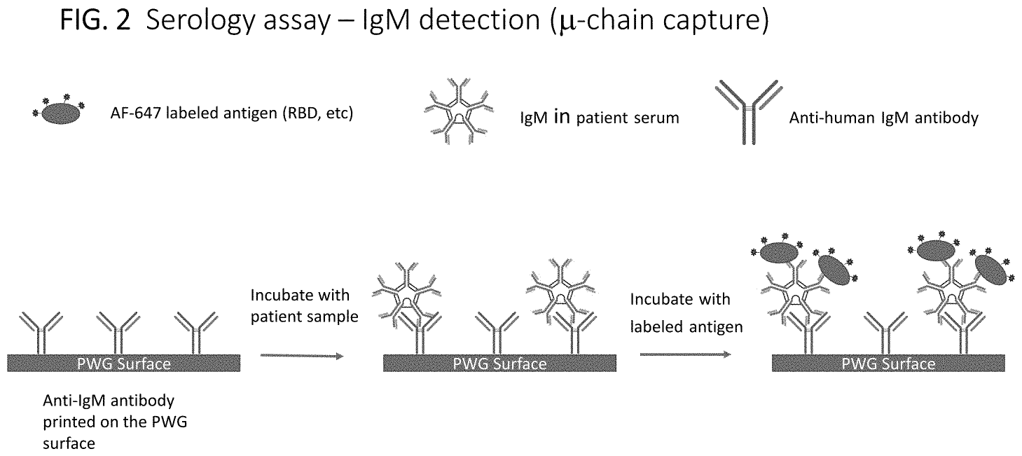

FIG. 3 illustrates a host response assay—protein biomarker detection using competition assay. In one example, anti-biomarker antibody is printed on the PWG surface. The surface is incubated with a patient sample diluted with labeled biomarker. A wash step may be used to remove excess patient sample. Subsequently, level of biomarker may be detected by measuring the fluorescence on the surface. The signal intensity obtained from the surface may be inversely proportional to the amount of biomarker in the sample.

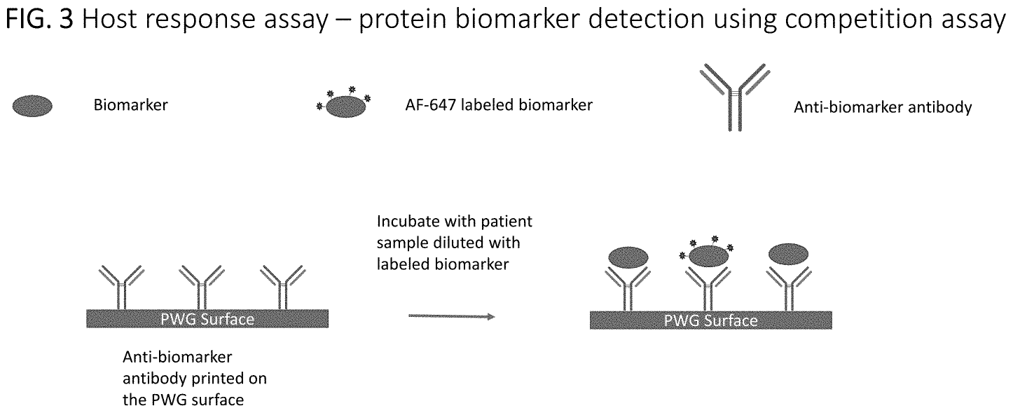

FIG. 4 illustrates a host response assay—protein biomarker detection using sandwich assay. In one example, anti-biomarker antibody is printed on the PWG surface. The surface is incubated with a patient sample diluted with labeling agent (such as anti-biomarker antibody—

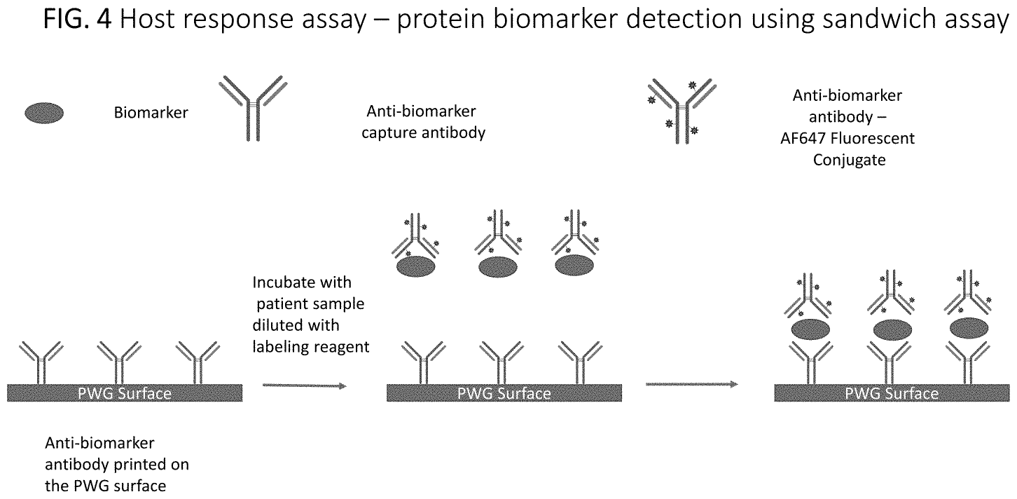

AF647 Fluorescent Conjugate). A wash step may be used to remove excess patient sample. Subsequently, level of biomarker may be detected by measuring the fluorescence on the surface.

FIG. 5 illustrates a serology assay—total Ig detection. In one example, SARS-CoV-2 antigen is printed on the PWG surface. The surface is incubated with a patient sample and labeled antigen. Then, a wash step may be used to remove excess patient sample and labeled antigen. Subsequently, level of total antibody in the patient sample can be detected by measuring the fluorescence on the surface.

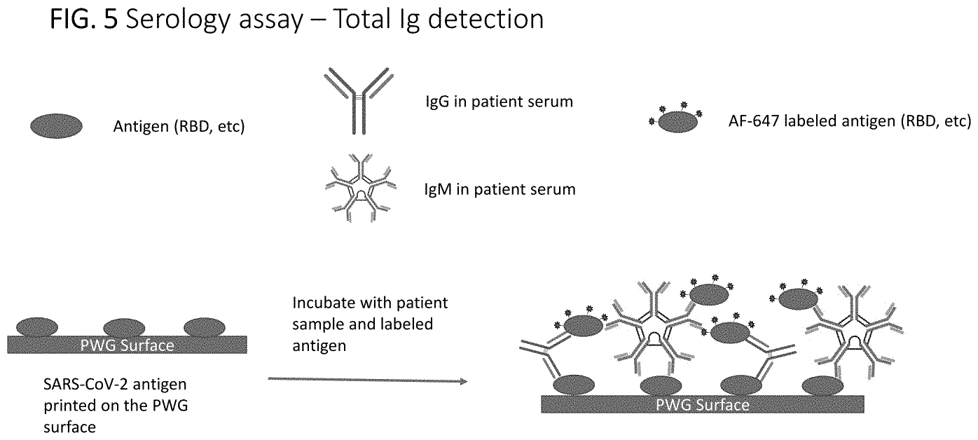

FIGS. 6A-6E illustrate a lateral flow version of the present disclosure.

FIGS. 7A-7C illustrate a direct competitive assay, in accordance with an embodiment.

FIG. 8 shows a flow chart illustrating one of the competitive assay processes, in accordance with an embodiment.

FIG. 9 illustrates antigenic peptides of SARS-CoV-2. In some embodiments, antigens in IgM/IgG test kits include at least one member selected from the group consisting of receptor binding domain (RBD) of the Spike (S) peptide, S1, S2, S1+S2 peptides of Spike, Nucleocapsid (N) peptides, and any combination thereof. In another embodiment, other SARS-CoV-2 antigens with diagnosis potential include Envelope (E) peptide and 3C-like Proteinase.

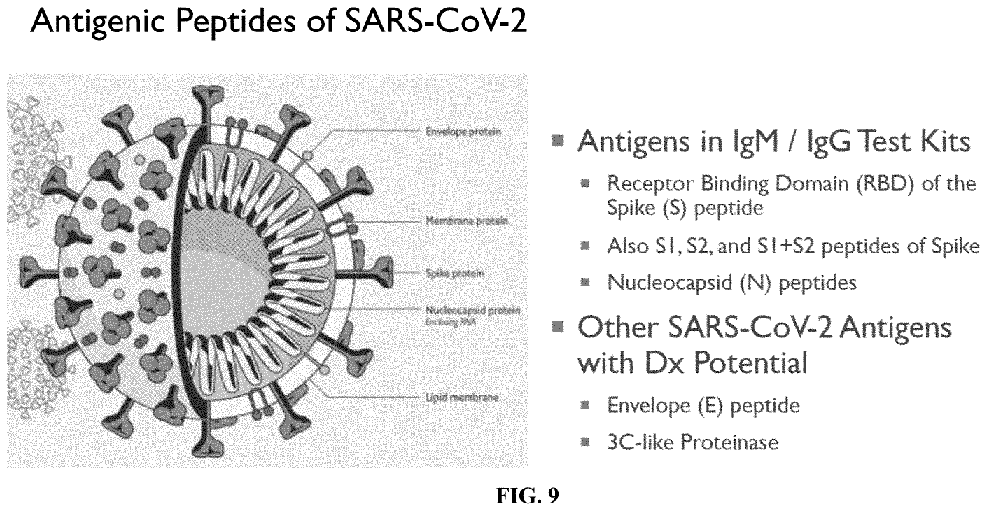

FIG. 10 illustrates Sensitivities of CRP, Fever, and Cough in Nonsevere COVID-19 Patients. In one example, Cumulative sensitivity of three tests simultaneously measured in POC is higher than 66%.

FIG. 11 illustrates implementation of some embodiments in the present disclosure. In one example. a sample from a subject is added to an IgG/IgM/CRP test. Then, the test is loaded to a Pearl-T Analyzer Connected to laptop running LightDeck® Studio Software. The patient information, including patient name, patient ID, and patient DOB, may be entered at the laptop or any appropriated device. In one example, the implementation may include checking body temperature of the subject and/or checking whether subject coughs.

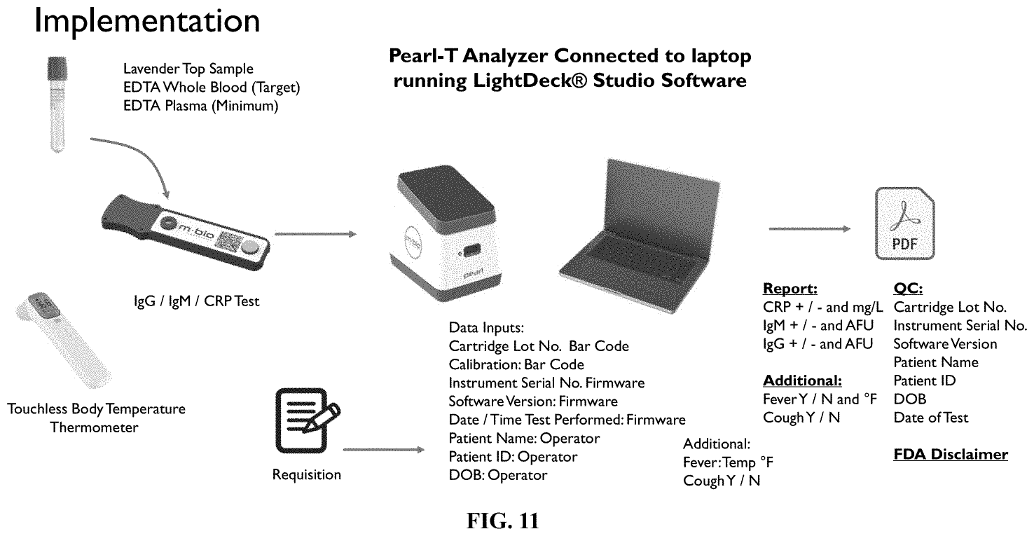

FIG. 12 illustrates some embodiments of the present disclosure. In one embodiment, the threshold level of CRP is 10 mg/L. In one embodiment, the threshold level of IgG/IgM is AFU at 3 SD above blank. In one embodiment, the threshold level of body temperature is 99.5° F. In one example, the level of CRP of the sample from a subject is lower than 10 mg/L, the level of IgG and IgM is above the threshold, the body temperature of the subject is lower than 99.5° F., and the subject does not cough, which in combination indicates the subject is ok to resume normal routine, and the subject is protected from (re-)infection. In another example, the level of CRP of the sample from a subject is lower than 10 mg/L, the body temperature of the subject is lower than 99.5° F., the subject does not cough, and the level of IgG and IgM is below the threshold, which in combination indicates the subject is ok to resume normal routine, however the subject is not protected from (re-)infection. In another example, the level of CRP of the sample from a subject is higher than 10 mg/L, the body temperature of the subject is higher than 99.5° F., the subject coughs, and the level of IgG and IgM is below the threshold, which in combination indicates the subject should not resume normal routine, and the subject is not protected from (re-)infection. In another example, the level of CRP of the sample from a subject is higher than 10 mg/L, the body temperature of the subject is higher than 99.5° F., and the subject coughs, the level of IgG and IgM is above the threshold, which in combination indicates the subject should not resume normal routine, and the subject may be protected from reinfection.

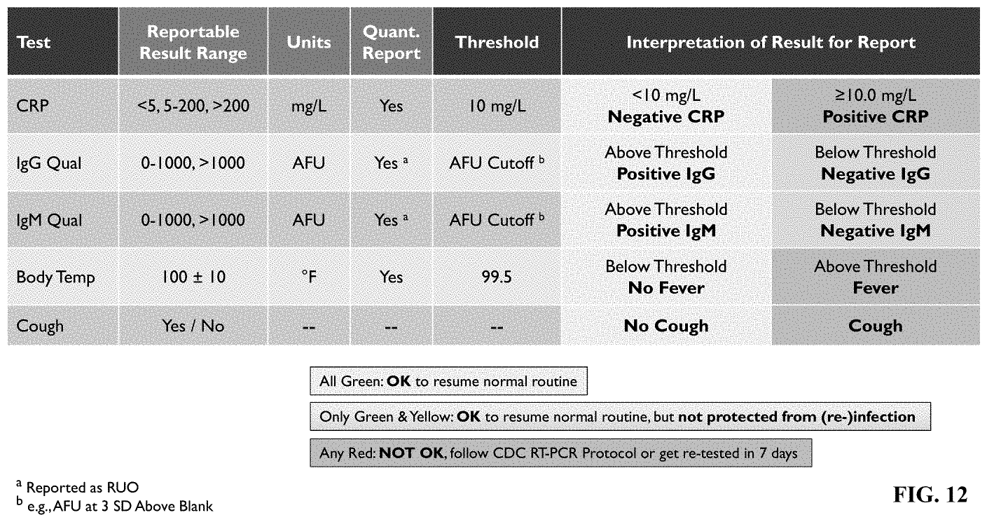

FIG. 13 illustrates one embodiment of a report of the present disclosure. In one embodiment, different colors may be used to indicate various conditions. For example, green indicates that the subject is ok to resume normal routine, and the subject is protected from (re-)infection, yellow for IgG/IgM report indicates that the subject is not protected from (re-) infection, and red for at least one of inflammatory biomarker (such as CRP), fever and cough indicates that the subject should not resume normal routine.

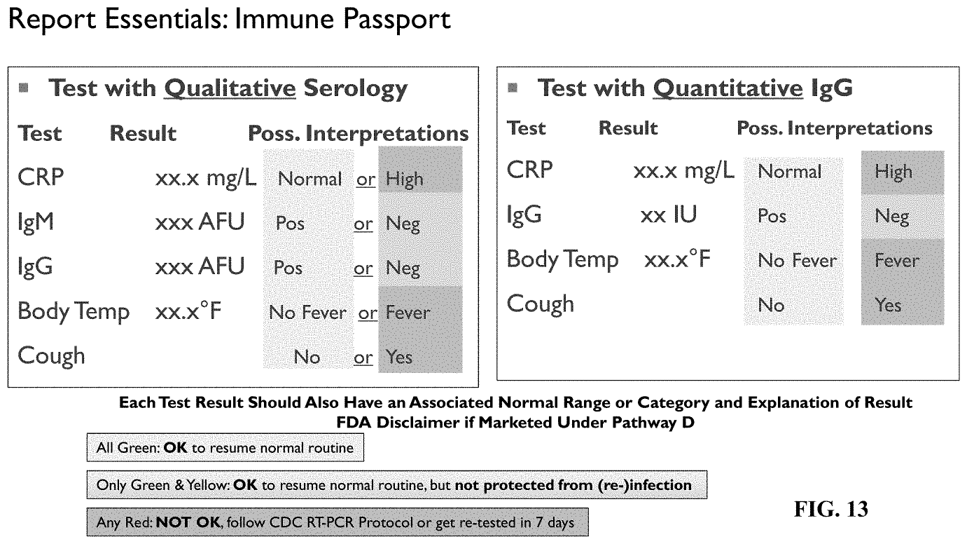

### DETAILED DESCRIPTION

The present disclosure provides a rapid, easily administered blood test to determine whether a subject has immune protection against a viral infection and whether he/she has an active infection and may spread the virus to other individuals. In one embodiment, the subject has previously tested positive for the infection. In one embodiment, the infection is a viral infection. In one aspect, the viral infection is caused by SARS-CoV-2. Wu et al., Nature 579 (7798), 265-269 (2020). In another embodiment, the present disclosure provides a method of combining a point of care (POC) serological test for antibodies against the pathogen with a POC test to determine whether the subject's immune response has returned to a normal level. In another embodiment, these two tests may be both performed at a point of care, such as a clinic or a hospital, at an airport, or at a port of entry. In another embodiment, these two tests may be performed from a single blood sample with one readout.

In one aspect, the present disclosure provides a method for (1) detecting qualitatively and/or quantitatively an antibody in a sample from the subject that binds specifically with an epitope of the pathogen, and (2) detecting and quantitating the level of one or more inflammatory biomarkers in the sample from the subject. In another aspect, tests (1) and (2) may be performed simultaneously in a multiplex assay using one single sample.

In one embodiment, the pathogen is a virus. In another embodiment, the inflammatory biomarker is one member selected from the group consisting of interleukin-1, interleukin-6, tumor necrosis factor α (TNFα), C-reactive protein (CRP), procalcitonin, ferritin and combination thereof. In respiratory viral infections, the host response is indicated by a rapid rise in these biomarkers post infection. These inflammatory markers play a necessary role to enable a concerted immunological response against the virus, including the generation of virus-specific immunoglobulins. At the end of an active infection, certain biomarkers of active infection decrease within 24 hours, while other biomarkers may increase. See Gong et al., medRxiv 2020.02.25.20025643.

In one aspect, the present disclosure provides a method for determining state of viral infection in a subject, wherein the subject has been tested positive for infection by a virus, said method comprising:

(a) detecting level of an antibody in a first sample from the subject, said antibody binding specifically with an epitope of the virus,

(b) comparing the level of the antibody to a predetermined antibody threshold level;

(c) detecting and quantitating level of at least one inflammatory biomarker in a second sample from the subject, and

(d) comparing the level of the inflammatory biomarker with a predetermined biomarker threshold level;

wherein, when the level of the antibody is higher than the predetermined antibody threshold level indicates that the subject has immune protection against the viral infection and when the level of the at least one inflammatory biomarker based on a predetermined biomarker level indicates that the subject does not have an active viral infection.

In one embodiment, when the level of one or more inflammatory biomarkers is higher than or equal to the predetermined biomarker, it indicates that the subject has an active infection. Examples of such biomarkers include, but are not limited to, Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

In another embodiment, the level of the at least one inflammatory biomarker lower than or equal to the predetermined biomarker indicates that the subject has an active infection. Examples of such biomarkers include, but are not limited to, Albumin, Transferrin, Transthyretin, and Retinol-binding protein.

In one embodiment, the inflammatory biomarker comprises at least one member selected from the group consisting of Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Albumin, Transferrin, Transthyretin, Retinol-binding protein, Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

In another embodiment, the inflammatory biomarker comprises at least two members selected from the group consisting of Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Albumin, Transferrin, Transthyretin, Retinol-binding protein, Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

In one embodiment, steps (a)-(d) are all performed at a point of care (POC) location. In another embodiment, steps (a) and (c) are preformed at POC and steps (b) and (d) may be performed off-site. In another embodiment, the first sample and second sample are the same sample obtained from the same subject. In another embodiment, the first sample and second sample are two different samples but steps (a) and (c) are performed at the same POC location during the same visit by the subject. In another embodiment, steps (a) and (c) are performed using the same device or instrument. In another embodiment, steps (a) and (c) are performed using two different devices or instruments. In another embodiment, steps (a) and (c) are performed simultaneously. In another embodiment, results from the comparing steps of (b) and (d) are presented on one single readout. In another embodiment, results from the comparing steps of (b) and (d) are presented on two or more readouts. In another embodiment, the method further includes a step of determining whether it is safe to release the subject from isolation or quarantine, wherein a decision to release the subject from isolation or quarantine requires both (1) the level of the antibody in the subject is higher than the predetermined antibody threshold level, and (2) the level of at least one inflammatory biomarker is lower than the predetermined biomarker level.

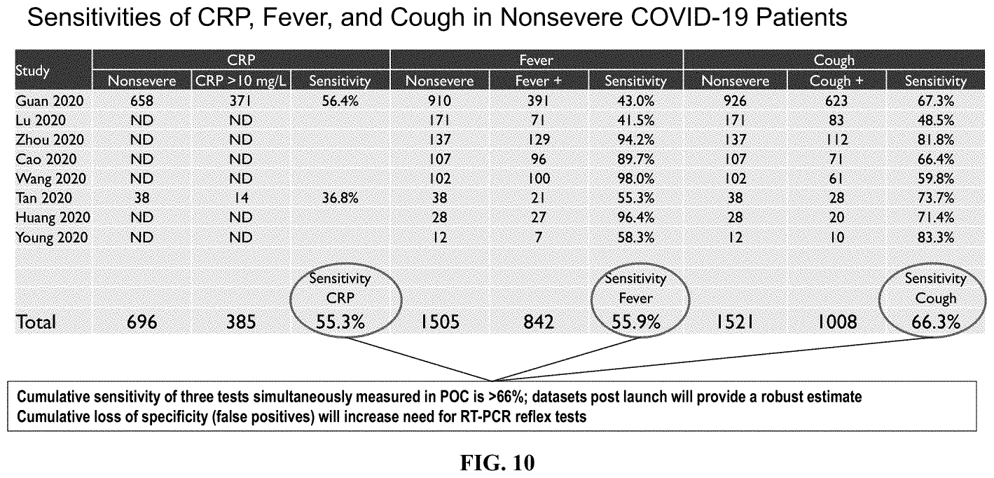

In another embodiment, the method further comprises a step of measuring the body temperature of the subject. In one aspect, when the subject's temperature is not higher than a predetermined temperature, it indicates that the subject does not have an active infection. In another embodiment, the temperature is measured with an infra-red touchless technology. In another embodiment, the method further comprises a step of checking whether the subject coughs. In another embodiment, the method further comprises a step of checking whether the subject has non-productive cough (dry cough). In another embodiment, the temperature and/or whether the subject coughs is measured prior to any of steps (a)-(d). Whether or not a subject coughs and/or has an elevated body temperature may be used in conjunction with inflammatory biomarker test to indicate whether the subject is still having active inflammation and may spread the pathogens to others (See FIG. 10).

In one embodiment, the inflammatory biomarker comprises at least two members selected from the group consisting of interleukin-1, interleukin-6, tumor necrosis factor α (TNFα), C-reactive protein (CRP), procalcitonin, ferritin, and combination thereof, and indication that the subject does not have an active viral infection requires that the level of the at least two inflammatory biomarker(s) is lower than their corresponding predetermined biomarker level.

In one embodiment, the antibody binds specifically to a viral antigen from a coronavirus. In one embodiment, the antibody binds specifically to an antigen from SARS-CoV-2, but not to viral antigens from other respiratory viruses. In one embodiment, the sample is selected from the group consisting of urine, blood, plasma and serum. In one embodiment, detecting the level of an antibody, and detecting and quantitating the level of an inflammatory biomarker are performed by a multiplex immunoassay.

In one embodiment, the antibody is of a subtype selected from the group consisting of IgM, IgG and IgA. In one embodiment, the antibody includes at least two subtypes selected from the group consisting of lateral flow version. In another embodiment, the epitope is located on the receptor binding domain (RBD), S1, S2 or N protein of SARS-CoV-2. In one aspect, the epitope is located on a protein that shares at least 70%, 80%, 90%, 95%, 99%, 99.5% sequence identity with RBD, S1, S2 or N protein of SARS-CoV-2. See Wu et al., Nature 579 (7798), 265-269 (2020).

In another aspect, the present disclosure provides a device for analyzing a sample, the device comprising: a) a planar waveguide; b) a refractive volume for optically coupling light provided by a light source to the planar waveguide; and c) a plurality of capture molecules, wherein the planar waveguide and the refractive volume are integrally formed as a single piece, and wherein the planar waveguide includes a first surface and a second surface that is opposite from the first surface, wherein the plurality of capture molecules is immobilized to the first surface, wherein at least one of the plurality of capture molecule is capable of specifically binding an antibody of a virus, and at least another one of the plurality of capture molecule is capable of specifically binding an inflammatory biomarker.

In another aspect, the present disclosure provides a device for analyzing a sample potentially including at least one analyte, the device comprising: a) a planar waveguide; b) a refractive volume for optically coupling light provided by a light source to the planar waveguide; and c) a plurality of capture molecules, wherein the planar waveguide and the refractive volume are integrally formed as a single piece, and wherein the planar waveguide including a first surface and a second surface that is opposite from the first surface, the plurality of capture molecules being immobilized to the first surface, the first surface including an array, the array including a first reaction site and a second reaction site, the first reaction site including at least a capture molecule that is capable of specifically binding an antibody of a virus, and the second reaction site including at least capture molecule is capable of specifically binding an inflammatory biomarker.

In one embodiment, the capture molecule at the first reaction site includes an antibody against human IgM, IgG or IgA.

In another embodiment, the capture molecule at the second reaction site comprises an antibody against an inflammatory biomarker selected from the group consisting of interleukin-1, interleukin-6, tumor necrosis factor α (TNFα), C-reactive protein (CRP), procalcitonin, ferritin, and combination thereof.

In one embodiment, the level of the inflammatory marker is quantitated. By way of example, in the case of CRP, the assay reporting range is from 5-200 mg/L. In some embodiment, the assay reporting range is 10-200 mg/L. In some embodiment, the assay reporting range is 15-200 mg/L. Results reported as non-infectious when level is <5 mg/L, <10 mg/L, or 15 mg/L, possible infectious when level is 5-200 mg/L, 10-200 mg/L, or 15-200 mg/L, infectious when level is >200 mg/L.

In another embodiment, treatment scheme may be designed based on the results from the serology test and/or the inflammatory biomarker test. For example, a persistent elevated level of certain inflammatory biomarker may indicate over-reaction by the subject's immune system and that anti-inflammatory drugs (e.g., anti-IL-6, or anti-IL-6R) may be needed to calm down the immune response.

In another embodiment, the device further comprises a labeling molecule comprising a detectable tag and a polypeptide comprising a fragment of at least one protein selected from the group consisting of RBD, S1, S2 and N protein of SARS-CoV-2. In one aspect, the at least one protein may share at least 70%, 80%, 90%, 95%, 99%, 99.5% sequence identity with RBD, 51, S2 or N protein of SARS-CoV-2. See Wu et al., Nature 579 (7798), 265-269 (2020).

In one aspect, the present disclosure provides a device for determining state of viral infection in a subject, including a sample receiving portion; a first capture area in flow contact with the sample receiving portion, wherein the first capture area comprises an immobilized first capture ligand, the immobilized first capture ligand comprises a capture molecule that is capable of specifically binding an antibody of a virus; and a second capture area in flow contact with the sample receiving portion, wherein the second capture area comprises an immobilized inflammatory biomarker.

In one embodiment, the antibody that binds specifically with an epitope of the virus and the inflammatory marker are measured using a quantitative multiplex assay. By way of example, the system, device and methods as described in U.S. Pat. No. 8,586,347, which is incorporated herein by reference, may be used for performing such a multiplex assay. In another embodiment, the quantitative multiplex assay is a quantitative bead-based multiplex immunoassay.

In one aspect, the present disclosure provides integrated assay kits to simultaneously measure the antibody that binds specifically with an epitope of a corona virus and at least one of the host inflammatory biomarkers. In one embodiment, the assay kit provides a “one stop” to assess whether it is safe to release a subject who has tested positive for infection by a virus from isolation/quarantine. In one embodiment, the assay kit comprises a plurality detection/quantification tools specific for the antibody that binds specifically with an epitope of a corona virus and for each of the at least one of the host inflammatory biomarkers. The antibody and the inflammatory biomarkers may be detected by immunoassays or like technologies.

The detection/quantification tools may comprise labeling ligands of multiple types, each directed to the selective labeling of the antibody or a specific biomarker in the sample, for example, comprising enzymatic, fluorescent, or chemiluminescent labels for the quantification of target species. For example, the capture and/or labeling ligands may comprise antibodies (or fragments thereof), affibodies, aptamers, or other moieties that specifically bind to a selected target. The assay kit may further comprise labeled secondary antibodies, for example comprising enzymatic, fluorescent, or chemiluminescent labels and associated reagents.

In one embodiment, the assay kit comprises a solid support to which one or more individually addressable patches of capture ligands are present, wherein the capture ligands of each patch are directed to a specific target (the antibody or the host inflammatory biomarker) described herein. In another embodiment, individually addressable patches of absorbent or adsorbing material are present, onto which individual aliquots of sample may be immobilized. Solid supports may include, for example, a chip, wells of a microtiter plate, a bead or resin. The chip or plate of the kit may comprise a chip configured for automated reading, as is known in the art.

In another embodiment, the assay kits of the disclosure comprise reagents or enzymes which create quantifiable signals based on concentration dependent reactions with the target species in the sample. Assay kits may further comprise elements such as reference standards of the target to be measured, washing solutions, buffering solutions, reagents, printed instructions for use, and containers.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

The term “subject” or “patient” as used herein is intended to include animals. Examples of subjects include but are not limited to mammals, e.g., humans, apes, monkeys, dogs, cows, horses, pigs, sheep, goats, cats, mice, rabbits, rats, and transgenic non-human animals. In an embodiment, the subject is a human.

As used herein, the terms “host inflammatory biomarker”, “marker of inflammation”, “inflammatory marker”, “inflammatory biomarker”, and plurals and grammatical equivalents thereof refer to markers which may be detected in a sample, and which may be identified in a sample, which indicate the presence of, or level of, inflammation in the subject from which the sample was obtained. Markers of inflammation include both peptide and non-peptide markers; for example, markers of inflammation include, without limitation, interleukin-1, interleukin-6, tumor necrosis factor α (TNFα), C-reactive protein (CRP), procalcitonin, ferritin, and combination thereof. In one embodiment, increase of the inflammation maker is associated with an active viral infection.

The term “capture antibody” is intended to include an immobilized antibody which is specific for (i.e., binds, is bound by, or forms a complex with) one or more analytes of interest in a sample such as a cellular extract. In one embodiment, the capture antibody is restrained on a solid support in an array.

The term “label” or “detectable moiety” is used herein to denote a composition detectable by spectroscopic, photochemical, biochemical, immunochemical, chemical, or other physical means. Examples of labels are 32P, fluorescent dyes, electron-dense reagents, enzymes (e.g., as commonly used in an ELISA), biotin, digoxigenin, and haptens and

proteins or other entities which can be made detectable, e.g., by incorporating a radio label into the peptide or by being used to detect antibodies specifically reactive with the peptide.

### The

labels can be incorporated, for example, into antibodies and/or other proteins at any position. Any method known in the art for conjugating the antibody to the label can be employed, for example, using methods described in Hermanson, Bioconjugate Techniques 1996, Academic Press, Inc., San Diego. Alternatively, methods using high affinity interactions can achieve the same results where one of a pair of binding partners binds to the other, e.g., biotin and streptavidin. The proteins of the invention as described herein can be directly labeled as with isotopes, chromophores, lumiphores, chromogens, or indirectly labeled such as with biotin to which streptavidin in a complex with a fluorescent, radioactive, or other moiety that can be directly detected can then bind. Thus, a biotinylated antibody is considered a “labeled antibody” as used herein.

The term “antibody” as used herein refers to a polypeptide encoded by an immunoglobulin gene or immunoglobulin genes, or fragments thereof, which specifically bind and recognize an analyte (such as antigen). In some embodiments, antibodies disclosed herein are anti-human antibodies. In another embodiment, those anti-human antibodies are labeled. In another embodiment, those antibodies are antibodies to human IgG, those that are antibodies to human IgM, and those that are antibodies to human IgA. An example of a structural unit of immunoglobulin G (IgG antibody) is a tetramer. Each such tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively. Antibodies exist as intact immunoglobulins or as well-characterized fragments produced by digestion of intact immunoglobulins with various peptidases. Thus, for example, pepsin digests an antibody near the disulfide linkages in the hinge region to produce F(ab′)2, a dimer of Fab which itself is a light chain joined to VH-CHI by a disulfide bond. The F(ab′)2 dimer can be reduced under mild conditions to break the disulfide linkage in the hinge region, thereby converting the F(ab′)2 dimer into two Fab′ monomers. The Fab′ monomer is essentially an Fab with part of the hinge region (see, Paul (Ed.), Fundamental Immunology, Third Edition, Raven Press, N.Y. (1993)). While various antibody fragments are defined in terms of the digestion of an intact antibody, one of skill will appreciate that such fragments may be synthesized de nova either chemically or by utilizing recombinant DNA methodology. Thus, the term “antibody,” as used herein, also includes antibody fragments either produced by the modification of whole antibodies or by de nova synthesis using recombinant DNA methodologies such as single chain F_{V}.

The term “specifically (or selectively)” in reference to binding to an antibody, or “specifically (or selectively) immunoreactive with” or “having binding specificity for,” when referring to a protein, peptide, or antigen, refers to a binding reaction which is determinative of the presence of the protein, peptide, or antigen in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an

antibody that is selected for its specificity for a particular protein. For example, antibodies raised against a protein can be selected to obtain antibodies specifically immunoreactive with that protein and not with other proteins. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, N.Y. (1988) for a description
of immunoassay formats and conditions that can be used to determine specific immunoreactivity. In one embodiment, a specific or selective reaction will be at least twice the background signal or noise. In another embodiment, a specific or selective reaction will be more than 10 to 100 times background signal or noise.

The term “biological sample” or “sample” encompasses a variety of sample types obtained from an organism. The term encompasses bodily fluids such as blood, saliva, serum, plasma, urine and other liquid samples of biological origin, and solid samples, such as a nasopharyngeal swab, a biopsy specimen or tissue cultures or cells derived therefrom and the progeny thereof. In one embodiment, the biological sample will be a bodily fluid or tissue that contains detectable amounts of antibodies. The term encompasses samples that have been manipulated in any way after their procurement, such as by treatment with reagents, solubilization, sedimentation, or enrichment for certain components. The term encompasses a clinical sample, and also includes cells in cell culture, cell supematants, cell lysates, serum, plasma, other biological fluids, and tissue samples. Preferred biological samples are blood samples, plasma samples, and serum samples.

The term “solid support” is used herein to denote a solid inert surface or body to which an agent, such as an antibody or an antigen, that is reactive in any of the binding reactions described herein can be immobilized. The term “immobilized” as used herein denotes a molecularly based coupling that is not dislodged or de-coupled under any of the conditions imposed during any of the steps of the assays described herein. Such immobilization can be achieved through a covalent bond, an ionic bond, an affinity-type bond, or any other chemical bond.

“Multiplex” assays are analyses that simultaneously measure the levels of more than one analyte in a single sample.

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., 75, 80, 85, 90, 91, 92, 93, 94, 95, 96, 97, 98, 99, or 100%). One of skill will recognize that some variability will arise depending on the method and/or threshold of determining binding.

The term “capture molecule” is used here to describe any of a variety of molecules that could be attached to the surface for performing a useful assay. The capture molecules may be a peptide, a polypeptide, a protein, an antibody, an antigen, an aptamer, a polysaccharide, a sugar molecule, a carbohydrate, a lipid, an oligonucleotide, a polynucleotide, a synthetic molecule, an inorganic molecule, an organic molecule, and combination thereof.

The terms “polypeptide,” “peptide” and “protein” may be used interchangeably in this disclosure. The terms “oligonucleotide,” and “polynucleotide” may also be used interchangeably in this disclosure. For purpose of this disclosure, when referring to a polypeptide or a polynucleotide molecule, it is intended that either the full length molecule or a fragment of the full length molecule may be used. Moreover, any mutated forms of a polypeptide (antigen) or the DNA molecule encoding such a polypeptide are also within the scope of the disclosure, if such mutation or mutations do not reside within any epitope of the polypeptide (antigen), or if the mutation or mutations do not substantially decrease the binding affinity between the polypeptide (antigen) and a specific antibody against the polypeptide or a fragment thereof. Plural or singular forms of a noun may be used interchangeably unless otherwise specified in the disclosure. Capture molecules may also be in the form of a molecular mixture. For example, a cell lysate preparation containing a mixture of molecules may be attached to the surface.

The term “pathogen” or “infectious agent” is used herein to refer to any disease-causing virus, bacteria, fungi, protozoa, or parasite that infects and causes disease in a subject.

The term “incubating” is used synonymously with “contacting” and “exposing” and does not imply any specific time or temperature requirements unless otherwise indicated.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1

This example pertains to a lateral flow version of the test. Lateral flow assays can be designed for visual read out (qualitative results) without an instrument.

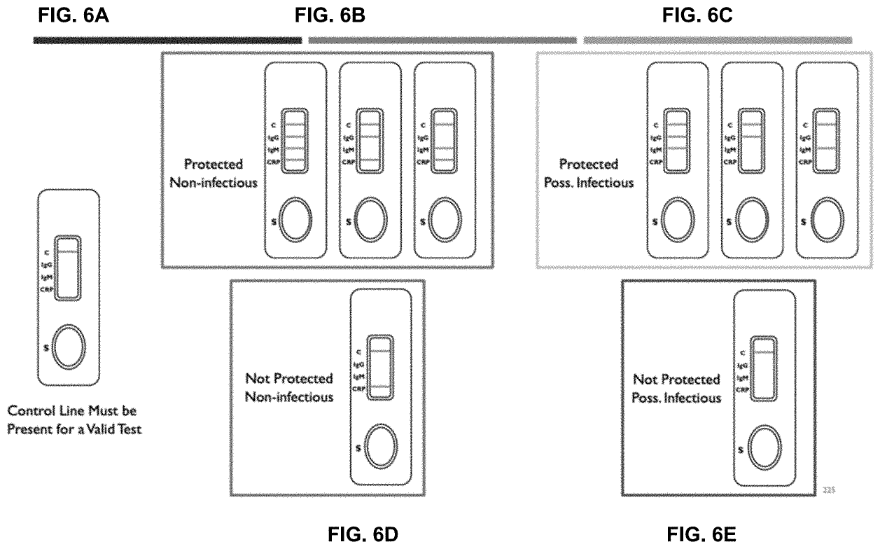

As shown in FIG. 6A-6E, the test strip includes a Control (C), which indicates whether the test passes or fails. If fail, end report. If pass, report IgM, IgG and CRP results. The test strip further includes indicators for IgG, IgM, and CRP (an example of a host response inflammatory marker). The predetermined CRP threshold level is 10 mg/L. For a lateral flow version of the test using a competitive fluorescent assay, this will be at just the point where the line is no longer visible to the average user.

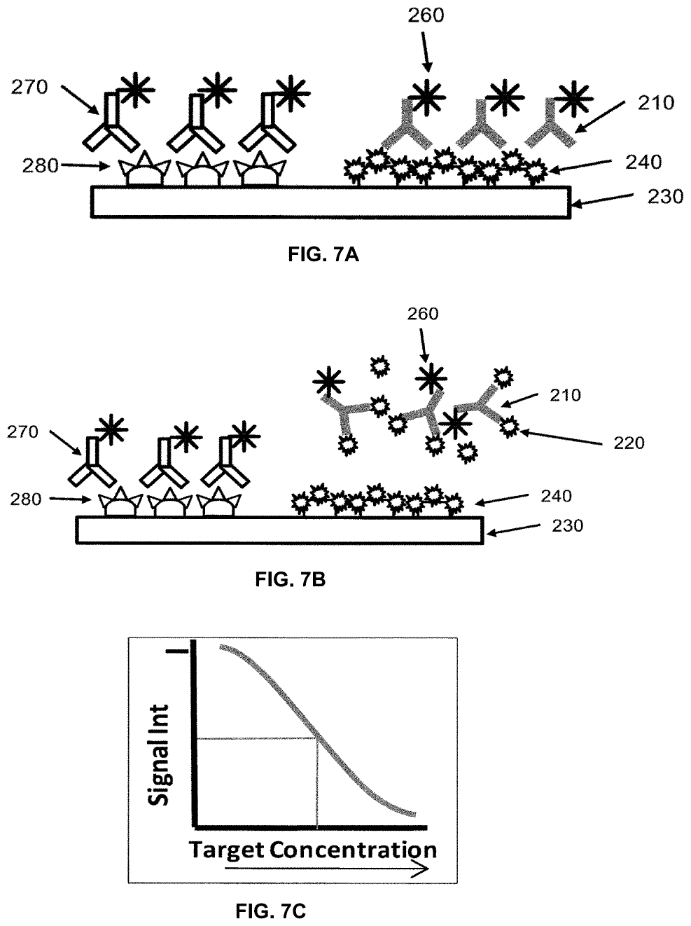

FIG. 7A-7C shows a diagrammatic representation of a direct competitive assay technique, in accordance with an embodiment of the present disclosure for detecting and/or quantitating an inflammatory biomarker and/or an antibody. According to FIGS. 7A-7B, a primary anti-CRP antibody is used as the labeling molecule 210, which may be mixed with a sample containing a target analyte (CRP) 220. A device having a surface 230 serves as the platform for the assay. Capture molecules 240, which are also CRP, are immobilized on the surface 230. In one embodiment, surface 230 may be a test strip or a waveguide. In another embodiment, surface 230 may be a planar waveguide having a refractive volume which optically couples light to the planar waveguide. By way of example, FIG. 7A shows CRP (same as target analyte) as the capture molecule 240. The labeling molecule 210 (anti-CRP antibody) is pre-conjugated with an excitable tag 260. When exciting light is shed on a spot on the surface 230, the excitable tag 260 emits light signal having intensity that is proportional to the amount of excitable tags attached to the spot. When no target analyte CRP present in the sample, all of the anti-CRP antibodies 210 bind to the capture molecule 240 (FIG. 7A). When target analyte CRP 220 is present in the sample, target analyte CRP 220 competes against capture molecule 240 in binding with the labeling molecules 210, thereby reducing the amount of labeling molecules 210 that are attached to the capture molecule 240 (FIG. 7B). Thus, the signal intensity obtained from the spot may be inversely proportional to the amount of target analyte in the sample (FIG. 7C).

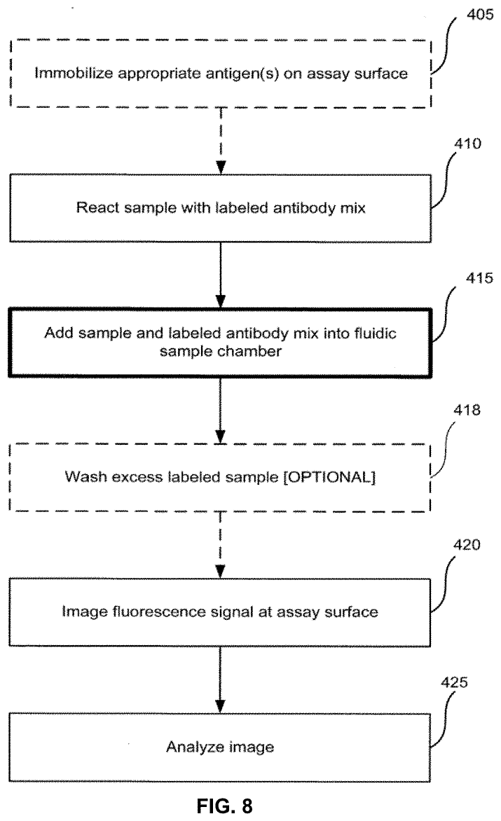

FIG. 8 shows a flow chart, summarizing an exemplary competitive assay process flow, in accordance with an embodiment. An assay process may begin with an antigen immobilization step 405, in which one or more appropriate antigens as well as potentially positive and negative controls are immobilized on an assay surface.

Assay process then proceeds to a step 410, in which a sample, and a labeled detect reagent mix is added to a fluidic sample chamber. The labeled antibody mix may be provided by the assay system manufacturer or custom-formulated by the assay system user. In step 415 the pre-mix of sample and labeled antibody created in step 410 may be added to the sample chamber. Optionally, excess detect reagent mix may be washed away from assay surface in an optional step 418. The fluorescence signal at the assay surface is then imaged by the assay system in a step 420, and then the captured image may be analyzed in a step 425.

All three samples in FIG. 6B include CRP below the predetermined CRP threshold level, which indicates that each subject for each of the three samples possibly is not infectious. In FIG. 6B, one sample includes both IgG and IgM based on a cut off fluorescence signal, one sample includes only IgG, one sample includes only IgM, which indicates that each subject for each of the three samples is protected from the virus and not infectious. Therefore, it is safe to release these subjects from isolation or quarantine.

All three samples in FIG. 6C include CRP above the predetermined CRP threshold level, which indicates that each subject for each of the three samples is possibly infectious. In FIG. 6C, one sample includes both IgG and IgM based on a cut off fluorescence signal, one sample includes only IgG, one sample includes only IgM, which indicates that each subject for each of the three samples is protected from the virus. Therefore, it is not safe to release these subjects from isolation or quarantine because they may still infect others.

In FIG. 6D, the sample does not include IgG or IgM, and includes CRP below the predetermined CRP threshold level, which indicates that the subject is not protected from the virus, and is not infectious. Therefore, it is not safe to release these subjects from isolation or quarantine because they are not protected and may be infected if they are exposed to the virus.

In FIG. 6E, the sample does not include IgG or IgM, and includes CRP above the predetermined CRP threshold level, which indicates that the subject is not protected from the virus, and is possibly infectious. Therefore, it is not safe to release these subjects from isolation or quarantine because they may infect others.

We claim:

1. A method for determining a subject's state of infection by a pathogen, said method comprising:
(a) measuring level of an antibody in a first sample from the subject, said antibody binding specifically with an epitope of the pathogen,
(b) comparing the level of the antibody to a predetermined antibody threshold level;
(c) measuring level of at least one inflammatory biomarker in a second sample from the subject, and
(d) comparing the level of the inflammatory biomarker with a predetermined biomarker threshold level;
wherein, when the level of the antibody is higher than the predetermined antibody threshold level indicates that the subject has immune protection against the pathogen, and the level of the at least one inflammatory biomarker(s) based on the predetermined biomarker threshold level indicates whether the subject does not have an active infection caused by the pathogen.

2. The method of claim 1, wherein said steps (a)-(d) are performed at a point of care (POC) location.

3. The method of claim 1, wherein said first sample and second sample are the same sample.

4. The method of claim 1, wherein said steps (a) and (c) are all performed simultaneously.

5. The method of claim 1, further comprising a step of determining whether it is safe to release the subject from isolation or quarantine, wherein a decision to release the subject from isolation or quarantine requires both (1) the level of the antibody in the subject is higher than the predetermined antibody threshold level, and (2) the level of at least one inflammatory biomarker based on the predetermined biomarker level indicates that the subject does not have an active infection caused by the pathogen.

6. The method of claim 1, wherein the inflammatory biomarker comprises at least one member selected from the group consisting of Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Albumin, Transferrin, Transthyretin, Retinol-binding protein, Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

7. The method of claim 1, wherein the inflammatory biomarker comprises at least two member selected from the group consisting of Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Albumin, Transferrin, Transthyretin, Retinol-binding protein, Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

8. The method of claim 1, wherein level of the inflammatory biomarker lower or equal to the predetermined biomarker threshold level indicates that the subject does not have an active infection.

9. The method of claim 8, wherein the inflammatory biomarker comprises at least one member selected from the group consisting of Interleukin 1 (IL-1), Interleukin 6 (IL-6), Interleukin 8 (IL-8, CXCL8), Interleukin 12 (IL-12), Interleukin 18 (IL-18), Tumor Necrosis Factor alpha (TNF-α), Interferon Gamma (IFNγ), Granulocyte-Macrophage Colony Stimulating Factor (GM-CSF), C-X-C motif chemokine 10 (CXCL10, IP-10), C-C chemokine ligand 3 (CCL3), Monocyte Chemoattractant Protein 1 (MCP1, CCL2), Monocyte Chemoattractant Protein 4 (MCP4), Macrophage-Derived Chemokine (MDC, CCL22), C-reactive protein (CRP), Serum Amyloid A (SAA), Haptoglobin (Hp), Ceruloplasmin, α2-Macroglobulin, α1-Acid glycoprotein (AGP), Fibrinogen, Complement (C3, C4), Heat shock protein 70 kDa 1B (HSPA1B), Granzyme B (GZMB), Matrix metallopeptidase 8 (MMP8), Procalcitonin (PCT), Ferritin, Von Willebrand Factor A2 (vWF A2), Vascular endothelial growth factor (VEGF), Tumor Necrosis Factor Receptor 1 (TNFR1, CD120a), Lipocalin-2 (LCN-2, NGAL), Soluble Intercellular Adhesion Molecule 1 (sICAM-1), Interleukin 1 Receptor Antagonist (IL-1 Ra), Soluble Receptor for Advanced Glycosylation (sRAGE), and Fatty Acid-Binding Protein 1 (FABP1, LFABP).

10. The method of claim 1, wherein level of the inflammatory biomarker higher or equal to the predetermined biomarker threshold level indicates that the subject does not have an active infection.

11. The method of claim 10, wherein the inflammatory biomarker comprises at least one member selected from the group consisting of Albumin, Transferrin, Transthyretin, and Retinol-binding protein.

12. The method of claim 1, wherein the antibody binds specifically to a viral antigen from a coronavirus.

13. The method of claim 1, wherein the antibody binds specifically to an antigen from SARS-CoV-2, but not to viral antigens from other respiratory viruses at a detectable level.

14. The method of claim 1, wherein the sample is selected from the group consisting of urine, saliva, blood, plasma and serum.

15. The method of claim 1, wherein detecting level of an antibody, and detecting and quantitating level of an inflammatory biomarker are performed by a multiplex immunoassay.

16. The method of claim 1, wherein the antibody is of a subtype selected from the group consisting of IgM, IgG and IgA.

17. The method of claim 12, wherein the epitope of the coronavirus is located on a protein of the coronavirus selected from the group consisting of receptor binding domain (RBD), S1, S2 and N protein.

18. A device for analyzing a sample from a subject to determine the subject's state of infection by a pathogen, the device comprising: a) a planar waveguide; b) a refractive volume for optically coupling light provided by a light source to the planar waveguide; and c) a plurality of capture molecules, wherein the planar waveguide and the refractive volume are integrally formed as a single piece, and wherein the planar waveguide comprises a first surface and a second surface that is opposite from the first surface, wherein the plurality of capture molecules is immobilized to the first surface, the plurality of capture molecules being immobilized to the first surface forming an array of at least two reaction sites: a first reaction site and a second reaction site, the first reaction site including at least a capture molecule that is capable of specifically binding an antibody, and the second reaction site including at least one capture molecule capable of specifically binding an inflammatory biomarker.

19. The device of claim 18, wherein the capture molecule on the first reaction site comprises a protein or fragment thereof of SARS-CoV-2, said protein or fragment being selected from the group consisting of RBD, S1, S2 and N protein, and the capture molecule on the second reaction site comprises at least one antibody capable of specifically binding an inflammatory biomarker selected from the group consisting of interleukin-1, interleukin-6, tumor necrosis factor α (TNFα), C-reactive protein (CRP), procalcitonin, ferritin, and combination thereof.

20. A device for analyzing a sample obtained from a subject to determine the subject's state of infection by a pathogen, the device comprising: a) a planar waveguide; b) a refractive volume for optically coupling light provided by a light source to the planar waveguide; and c) a plurality of capture molecules, wherein the planar waveguide and the refractive volume are integrally formed as a single piece, and wherein the planar waveguide including a first surface and a second surface that is opposite from the first surface, the plurality of capture molecules being immobilized to the first surface, the first surface including an array, the array including a first reaction site and a second reaction site, the first reaction site including at least a capture molecule that is capable of specifically binding an antibody, and the second reaction site including at least one capture molecule capable of specifically binding an inflammatory biomarker.

21. The device of claim 20, wherein the capture molecule at the first reaction site comprises an antibody against human IgM, IgG or IgA, wherein the sample is loaded onto the device before a labeling mix is loaded, wherein the labeling mix comprises a labeling molecule, the labeling molecule comprising a detectable tag and a protein or fragment thereof of SARS-CoV-2, said protein or fragment being selected from the group consisting of RBD, S1, S2 and N protein.

22. The device of claim 20, wherein the capture molecule at the second reaction site comprises an antibody against an inflammatory biomarker selected from the group consisting of interleukin-1, interleukin-6, tumor necrosis factor α (TNFα), C-reactive protein (CRP), procalcitonin, ferritin, and combination thereof.

23. A device for analyzing a sample obtained from a subject to determine the subject's state of infection by a pathogen, the device comprising:
a sample receiving portion;
a first capture area in flow contact with the sample receiving portion, wherein the first capture area comprises an immobilized first capture ligand, the immobilized first capture ligand comprises a capture molecule that is capable of specifically binding an antibody; and
a second capture area in flow contact with the sample receiving portion, wherein the second capture area comprises an immobilized inflammatory biomarker or an immobilized second capture ligand, the immobilized second capture ligand comprises a capture molecule that is capable of specifically binding an inflammatory biomarker.

24. The device of claim 23, further comprising a detectable tag conjugated with an antibody against the inflammatory biomarker, and a detectable tag conjugated with an antibody against human IgM, IgG or IgA.

25. The device of claim 23, wherein the first capture ligand comprises a protein or fragment thereof of SARS-CoV-2, said protein or fragment being selected from the group consisting of RBD, S1, S2 and N protein.

26. A method for determining a subject's state of infection by a pathogen, said method comprising:
a) detecting presence or absence of an antibody in a first sample from the subject, said antibody binding specifically with an epitope of the pathogen, and
b) detecting one or more inflammatory biomarkers in a second sample from the subject, and
wherein, presence of the antibody indicates that the subject has immune protection against the viral infection and wherein the respective levels of the one or more inflammatory biomarkers being lower than a predetermined level for the respective inflammatory biomarkers indicates that the subject does not have an active viral infection.

27. The method of claim 26, wherein said first sample and second sample are the same sample.

28. The method of claim 26, wherein step (b) comprises
b1) Mixing the second sample with an antibody that specifically binds to one inflammatory biomarker,
b2) applying the mix from (b1) to a spot on a testing device, said spot having said inflammatory biomarker immobilized onto it,
b3) detecting presence or absence of said antibody at said spot to determine the level of the inflammatory biomarker.

29. The method of claim 28, wherein the antibody in step (b1) is labeled with a detectable tag.
